SEQUENCE LISTING (33 sequences)

SEQ ID NO: 1 - DNA, 20 bp, Synthetic construct
  source 1..20: mol_type = other DNA
tgccaatcgcgagcagcatc

SEQ ID NO: 2 - DNA, 20 bp, Synthetic construct
  source 1..20: mol_type = other DNA
aggttgcactcgtagtccag

SEQ ID NO: 3 - DNA, 17 bp, Synthetic construct
  source 1..17: mol_type = other DNA
cactgcaccgcctgggc

SEQ ID NO: 4 - DNA, 75 bp, Synthetic construct
  source 1..75: mol_type = other DNA
acaagcacagctacaccctgccaatcgcgagcagcattcagctgcaggtatggtggctggggttgcgcagccagt

SEQ ID NO: 5 - DNA, 75 bp, Synthetic construct
  source 1..75: mol_type = other DNA
gggccagttcaactttgtccacgtgatcgtcaccctgctggattacgagtgcaacctggtgtccctgcagtgcag

SEQ ID NO: 6 - DNA, 80 bp, Synthetic construct
  source 1..80: mol_type = other DNA
cttcctgacaggcctttgacttcctgttgctgctgtgggccgattcactgcaccgcctgggcctgcccaacaaggatgga

SEQ ID NO: 7 - RNA, 20 nt, Synthetic construct
  source 1..20: mol_type = other RNA
aaatgtgagatcagagtaat

SEQ ID NO: 8 - DNA, 20 bp, Synthetic construct
  source 1..20: mol_type = other DNA
tctgcagaccaagctgtaca

SEQ ID NO: 9 - DNA, 20 bp, Synthetic construct
  source 1..20: mol_type = other DNA
gtccttttctctgccccaac

SEQ ID NO: 10 - DNA, 20 bp, Synthetic construct
  source 1..20: mol_type = other DNA
tttgctgctgtggagagaga

SEQ ID NO: 11 - DNA, 19 bp, Synthetic construct
  source 1..19: mol_type = other DNA
ctctgcagcttccaggaac

SEQ ID NO: 12 - DNA, 20 bp, Synthetic construct
  source 1..20: mol_type = other DNA
ctgggtttgaaggtcgtgtg

SEQ ID NO: 13 - DNA, 20 bp, Synthetic construct
  source 1..20: mol_type = other DNA
acagatgtgtggactgcagg

SEQ ID NO: 14 - DNA, 20 bp, Synthetic construct
  source 1..20: mol_type = other DNA
tagaggtgtcttgcctgtgg

SEQ ID NO: 15 - DNA, 20 bp, Synthetic construct
  source 1..20: mol_type = other DNA
atgtctggggagacttggtg

SEQ ID NO: 16 - DNA, 20 bp, Synthetic construct
  source 1..20: mol_type = other DNA
tagccgagatcagccttcag

SEQ ID NO: 17 - DNA, 20 bp, Synthetic construct
  source 1..20: mol_type = other DNA
ttgcggtcagacacgatctt

SEQ ID NO: 18 - DNA, 20 bp, Synthetic construct
  source 1..20: mol_type = other DNA
agctgacaggtgtctagcag

SEQ ID NO: 19 - DNA, 19 bp, Synthetic construct
  source 1..19: mol_type = other DNA
actcacatttgcgtgcagg

SEQ ID NO: 20 - DNA, 20 bp, Synthetic construct
  source 1..20: mol_type = other DNA
cagagcctgtgtctgtgttg

SEQ ID NO: 21 - DNA, 20 bp, Synthetic construct
  source 1..20: mol_type = other DNA
ccgctggtcttcttctcaga

SEQ ID NO: 22 - DNA, 20 bp, Synthetic construct
  source 1..20: mol_type = other DNA
ccttttctgagtgcctgtgg

SEQ ID NO: 23 - DNA, 20 bp, Synthetic construct
  source 1..20: mol_type = other DNA
gcaggaacggaacagacttg

SEQ ID NO: 24 - DNA, 20 bp, Synthetic construct
  source 1..20: mol_type = other DNA
gatgtgggtgtgtgcacatc

SEQ ID NO: 25 - DNA, 20 bp, Synthetic construct
  source 1..20: mol_type = other DNA
ggacatccctcagacatgca

SEQ ID NO: 26 - DNA, 20 bp, Synthetic construct
  source 1..20: mol_type = other DNA
ccactacaagcacagctaca

SEQ ID NO: 27 - DNA, 21 bp, Synthetic construct
  source 1..21: mol_type = other DNA
gccaacatctatagcgcaaac

SEQ ID NO: 28 - DNA, 21 bp, Synthetic construct
  source 1..21: mol_type = other DNA
attagaggtgtcttgcctgtg

SEQ ID NO: 29 - DNA, 19 bp, Synthetic construct
  source 1..19: mol_type = other DNA
ccaggttgcactcgtagtc

SEQ ID NO: 30 - DNA, 19 bp, Synthetic construct
  source 1..19: mol_type = other DNA
ggacaccaggctctgtgag

SEQ ID NO: 31 - DNA, 20 bp, Synthetic construct
  source 1..20: mol_type = other DNA
tcagagcctctctctggctc

SEQ ID NO: 32 - DNA, 18 bp, Synthetic construct
  source 1..18: mol_type = other DNA
tgtaaaacgacggccagt

SEQ ID NO: 33 - DNA, 20 bp, Synthetic construct
  source 1..20: mol_type = other DNA
tgcaaatggcaagcagcatc

(19) United States
(12) Patent Application Publication
(10) Pub. No.: US 2025/0354178 A1
(43) Pub. Date: Nov. 20, 2025

(54) CRISPR/Cas9-BASED BASE EDITING OF TUBEROUS SCLEROSIS COMPLEX 2 GENE IN MESENCHYMAL STEM CELLS

(71) Applicant: UNIVERSITY OF MARYLAND, BALTIMORE, Baltimore, MD (US)

(72) Inventors: Miroslaw JANOWSKI, Towson, MD (US); Abdallah SALEMDAWOD, Baltimore, MD (US); Piotr WALCZAK, Towson, MD (US)

(21) Appl. No.: 19/210,206

(22) Filed: May 16, 2025

Related U.S. Application Data
(60) Provisional application No. 63/648,332, filed on May 16, 2024.
(60) Provisional application No. 63/712,646, filed on Oct. 28, 2024.

(51) Int. Cl. C12N 15/90; C12N 9/22; C12N 15/11; C12N 15/88

(52) U.S. Cl. CPC C12N 15/907; C12N 9/226; C12N 15/111; C12N 15/88

(57) ABSTRACT

A method of inducing SNP mutations in mesenchymal stem cells (MSCs), targeting the most frequent SNP mutations of the TSC2 gene, TSC2.1864C>T (p.Arg622Trp), TSC2.1832 G>A (p.Arg611Glu), and TSC2.5024 C>T (p.Pro1675Leu) using delivery methods for CRISPR components, is described. A high editing efficiency (up to 85%) for inducing TSC2 SNP mutations in MSCs using lipofectamine-based transfection was achieved. Overall, the high editing efficiency of some TSC2 mutations enables the induction and reversal of mutations in primary hMSCs without requiring the resource-consuming derivation of cell lines that are frequently distinct from their primary counterparts.

### CROSS REFERENCE TO RELATED APPLICATIONS

This application is filed under the provisions of 35 U.S.C. § 111(a) and claims priority to U.S. Provisional Patent Application No. 63/648,332 filed on May 16, 2024 in the name of Miroslaw Janowski, et al., and entitled “Method for editing tuberous sclerosis 2 (TSC2) gene in human cells using CRISPR/Cas9 based editing system,” and to U.S. Provisional Patent Application No. 63/712,646 filed on Oct. 28, 2024 in the name of Miroslaw Janowski, et al., and entitled “CRISPR/Cas9-based base editing of tuberous sclerosis complex 2 gene in mesenchymal stem cells,” both of which are hereby incorporated by reference herein in their entirety.

### SEQUENCE LISTING

The text of the computer readable sequence listing filed herewith, titled “UMB_43980_202_SequenceListing.xml,” created May 15, 2025, having a file size of 30,263 bytes, is hereby incorporated by reference in its entirety.

### FIELD

The present invention relates to methods of using CRISPR-based base editing for single-nucleotide point (SNP) modifications in human mesenchymal stem cells (hMSCs). Advantageously, the methods described herein are precise and efficient and the modified hMSCs retain their multipotency and viability.

### DESCRIPTION OF THE RELATED ART

Tuberous sclerosis complex genes 1 (TSC1) and 2 (TSC2) are tumor suppressor genes that act as convergence points of a complex signaling cascade controlling several cellular functions. The TSC1 gene is located on chromosome 9 (9q34) and encodes the protein Hamartin, while the TSC2 gene is located on chromosome 16 (16p13.3) and encodes the protein Tuberin. Both proteins form a complex that senses cellular growth factor stimulation and conveys the signal to the mammalian target of rapamycin (mTOR), which in turn regulates critical cellular functions, including growth and proliferation. A loss-of-function mutation in either of these tumor suppressor genes leads to maladaptation and overactivation of the mTOR pathway, and promotes tumorigenesis. Among the 1500 reported mutations in TSC, 75% affected the TSC2 gene, which makes it a more compelling target. Approximately 85% of the reported mutations are SNP mutations [Caban et al., 2017].

Although the specific mechanism by which these mutations initiate pathogenesis remains unclear, overactivation of mTORC1 is caused by dysregulated upstream signaling through phosphoinositide 3-kinase (PI3K), PTEN, AKT, and/or TSC1-TSC2 is observed in cancers, hamartomatous syndromes, such as TSC, and vascular anomalies (FIG. 1). TSC1/2 mutations not only play a role in the pathomechanism of these tumors but also have a significant impact on their prognosis [Mehta et al., 2011; Wang et al., 2023; Chakraborty et al., 2008].

Among the several clinical manifestations of TSC1/2 mutations, structural epilepsy is the most prevalent and clinically challenging. While managing epilepsy associated with TSC remains challenging, early diagnosis and therapy have demonstrated a significant impact on long-term outcomes. Traditional treatment approaches involving antiepileptic drugs are still limited. The effect of TSC2 mutations on the progress and outcome of several cancer diseases also highlights the need to explore and potentially target these mutations as part of oncological treatment.

Overall, there is a compelling need for a better understanding of the impact of specific TSC2 mutations on pathological processes in a personalized fashion [Uysal et al., 2020; Slowinska et al., 2018]. This is particularly valuable in the context of a neonatal sequencing storm. Studies on MSCs may be of special value in predicting the severity of pathological phenotypes during the pre-symptomatic period. Moreover. Wharton's jelly and cord blood are readily available sources of mesenchymal stem cells (MSCs) to test pathological signatures of particular mutations in the context of the entire genomic landscape and the feasibility of mutation corrections in parallel with neonatal development [Gornicka-Pawlak et al., 2019].

There continues to be a need in the art for methods of delivery of CRISPR-Cas9 components to human MSCs (hMSCs), enabling substantial gene editing efficiency of TSC2 SNPs while simultaneously minimizing off-target editing and cell toxicity.

### SUMMARY

In some aspects, a method for generating genomically engineered cells is described, the method comprising:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) culturing the pool comprising modified cells to produce a cultured pool; and
(d) selecting genomically engineered cells from the cultured pool wherein at least 35% of cells in the cultured pool comprise a mutation in a Tuberous sclerosis complex 2 (TSC2) gene.

In some embodiments, the genomically engineered cells comprise at least one nucleotide point mutation.

In some other aspects, a method for correcting or reversing a pathogenic mutation in a TSC2 gene, the method comprising:
(a) providing a pathogenic cell comprising the pathogenic mutation at the TSC2 gene;
(b) introducing into the pathogenic cell a Cas component, a guide RNA (gRNA) directed to a gRNA target, and a corrective homology directed repair (HDR) template to produce a corrected cell,
   wherein the corrective HDR template comprises a nonpathogenic sequence of the TSC2 gene.

Different embodiments, features and advantages of the invention will be more fully apparent from the disclosure below and appended claims.

### BRIEF DESCRIPTION OF THE DRAWINGS

FIG. 1 illustrates the regulation of mTORC1 signaling involves intricate modulation by growth factors and insulin. Numerous positive and negative regulators influence mTORC1 activity. Growth factors indirectly activate mTORC1 by suppressing the inhibitory function of the TSC1/TSC2 complex. TSC2 harbors a GAP domain that converts Rheb to its inactive, GDP-bound state. Phosphorylation by PI3K-AKT inhibits the TSC1/TSC2 complex, thereby alleviating its repression of Rheb and permitting TORC1 activation. AKT also enhances mTORC1 activity by negatively phosphorylating the mTORC1 suppressor PRAS40. Additionally, FKBP38 binds to the FRB domain of mTOR, leading to its dissociation from mTORC1 and consequently promoting mTORC1 activation.

FIG. 2A shows the comparative analysis of cell viability (squares) and GFP expression (circles) across different transfection conditions. Lipofectamine 2000 serves as a reference standard, while CRISPRMAX was tested at three Cas9 reagent to lipofectamine optimized ratios −2.5:1.5 μL, 5:2.5 μL, and 6:3 μL. Statistical significance is indicated by p-values above the bars, demonstrating meaningful differences between conditions.

FIG. 2B is a representative fluorescence microscopy image validating the quantitative data for lipofectamine 2000 transfection showing characteristic GFP expression pattern with 71% transfection efficiency. The image presents merged brightfield and fluorescence channels, with cellular morphology in grayscale and GFP signal in green. Scale bars: 50 μm.

FIG. 2C is a representative fluorescence microscopy image validating the quantitative data for CRISPRMAX at 2.5:1.5 μL ratio displaying comparable GFP expression to the second bar (73%). The image presents merged brightfield and fluorescence channels, with cellular morphology in grayscale and GFP signal in green. Scale bars: 50 μm.

FIG. 2D is a representative fluorescence microscopy image validating the quantitative data for CRISPRMAX at 5:2.5 μL ratio corresponding to the third bar showing enhanced transfection efficiency (89%). The image presents merged brightfield and fluorescence channels, with cellular morphology in grayscale and GFP signal in green. Scale bars: 50 μm.

FIG. 2E is a representative fluorescence microscopy image validating the quantitative data for CRISPRMAX at 6:3 μL ratio relating to the fourth bar demonstrating optimized GFP expression (86%). The image presents merged brightfield and fluorescence channels, with cellular morphology in grayscale and GFP signal in green. Scale bars: 50 μm.

FIG. 3 illustrates CRISPR/Cas9 Indel distributions of various point mutations in various cell groups. The hatched bars represent editing efficiency for (P1 TSC2.1864C>T), achieving 59% score in HEK293 using electroporation, 47% using Lipofection in HEK293, and 43% in hMSCs. The white bars show an editing score of P2 (TSC2.1832 G>A), reaching 73% in HEK293 cells, 80% in hMSCs using (1 mg Cas9:0.5 mg gRNA ratio) and 85% after increase of gRNA to 0.75 mg per reaction. The black bars reflect an editing score of 51% using lipofectamine CRISPRMax in HEK293, 28% in hMSCs using (1 mg Cas9:0.5 mg gRNA) and 38% after increase if gRNA to 0.75 mg.

FIG. 4A illustrates the inference of CRISPR Edits (ICE) analysis, Synthego. The upper gDNA sequence showing point mutation P1 TSC2.1864C>T on position 235 and an unwanted edit on position 243 TSC21872C>T compared to the lower wildtype sequence.

FIG. 4B illustrates the inference of CRISPR Edits (ICE) analysis, Synthego. The upper sequence showing point mutation P2 TSC2.1832 G>A on position 460.

FIG. 4C illustrates the inference of CRISPR Edits (ICE) analysis, Synthego. The upper sequence showing point mutation P3 TSC2.5024 C>T on position 228.

FIG. 5A shows the Cell Counting Kit 8 (CCK8) assay for hMSCs transfected with CRISPR Cas9 and gRNA targeting point mutations P2, P3, and non-functional gRNA as wildtype control. In the 1st transfection, cells received 500 Cas9 RNP and gRNA ratio. The CCK8 assay showed a mean absorbance of 0.43 on day 3, 1.09 on day 7, and 1.27 on day 14 after transfection. Each cell group showed significant differences of the absorbance from day 3 to day 14. No significant differences were detected between the cell groups on any of the days.

FIG. 5B is a cell viability graph that shows a mean cell viability of 46.82% on the third day after transfection, 84.04% on day 7, and 92.33% on day 14. Similarly, ANOVA statistics shows significant differences in cell viability in each cell group over time after transfection; without significant differences when comparing the cell groups.

FIG. 6A shows the agarose gel electrophoresis showing positive cleavage detection in hMSCs with SNP mutation P2 TSC2.1832 G>A using 1^{st} (500 ng gRNA) and 2^{nd} (750 ng gRNA) runs, both compared to the control group.

FIG. 6B shows agarose gel electrophoresis showing positive cleavage detection in hMSCs with SNP mutation P3 TSC2.5024 C>T using 1^{st} (500 ng gRNA) and 2^{nd} (750 ng gRNA) runs, both compared to the control group.

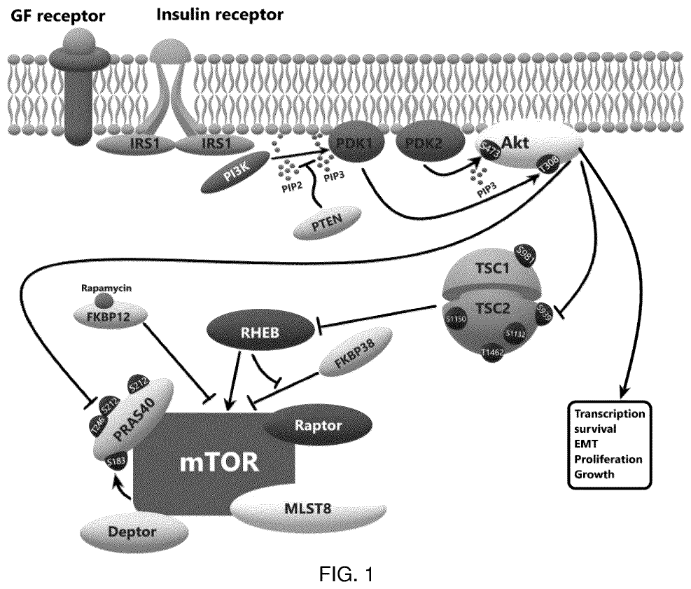

### DETAILED DESCRIPTION AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, “sample cells” or “cells” can include, but are not limited to, the cells described hereinafter. In some embodiments, cells are eukaryotic or prokaryotic cells. In some embodiments, cells are mammalian cells (e.g., human cells, canine cells, bovine cells, ovine cells, feline cells, or rodent cells such as rabbit, mouse, or rat cells, such as the Chinese Hamster Ovary (CHO) cells). In some embodiments, cells are expanded and/or differentiated for therapeutic use such as implantation into a subject (e.g., a human subject) in order to provide or supplement a cellular, tissue, or organ function that is missing or defective in the subject. In some embodiments, cells are human 293 cells (e.g., 293-T or HEK 293 cells), murine 3T3 cells, Chinese hamster ovary (CHO) cells, CML T1 cells, or Jurkat cells. In some embodiments, cells are primary cells, feeder cells, or stem cells. In some embodiments, cells are isolated from a subject (e.g., a human subject). In some embodiments, cells are primary cells isolated from a tissue or a biopsy sample. In some embodiments, cells are hematopoietic cells. In some embodiments, cells are stem cells, e.g., embryonic stem cells, mesenchymal stem cells, cancer stem cells, etc. In some embodiments, cells are isolated from a tissue or organ (e.g., a human tissue or organ), including but not limited to, solid tissues and organs. In some embodiments, cells can be isolated from placenta, umbilical cord, bone marrow, liver, blood, including cord blood, or any other suitable tissue. In some embodiments, patient-specific cells are isolated from a patient for culture (e.g., for cell expansion and optionally differentiation) and subsequent re-implantation into the same patient or into a different patient. In some embodiments, the cells may be genetically modified, expanded and reintroduced into a patient for the purpose of providing an immunotherapy (e.g., chimeric antigen receptor (CAR) cells for CAR-therapy (CAR-T), or delivery of CRISPR-Cas modified cells). In some embodiments, a primary cell culture includes epithelial cells (e.g., corneal epithelial cells, mammary epithelial cells, etc.), fibroblasts, myoblasts (e.g., human skeletal myoblasts), keratinocytes, endothelial cells (e.g., microvascular endothelial cells), neural cells, smooth muscle cells, hematopoietic cells, placental cells, or a combination of two or more thereof. In some embodiments, cells are recombinant cells (e.g., hybridoma cells or cells that express one or more recombinant products).

“Complementarity” refers to the ability of a nucleic acid to form hydrogen bond(s) with another nucleic acid sequence, e.g., by traditional Watson-Crick base pairing. A percent complementarity indicates the percentage of residues in a nucleic acid molecule which can form hydrogen bonds with a second nucleic acid sequence (e.g., 50%, 60%, 70%, 80%, 90%, or 100% complementary).

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “gRNA” is used herein to refer to a guide RNA sequence selected to specifically target a particular nucleic acid sequence of interest, hereinafter referred to as a “gRNA target.” Complexing between a Cas polypeptide and a gRNA can include the binding of the gRNA and polypeptide by covalent bonding, hydrogen bonding, and/or other non-covalent bonding, and is well-understood in the field of CRISR-Cas systems. Exemplary gRNAs can therefore comprise a section that is targeted for a particular nucleic acid sequence of interest and section that is for binding to the Cas polypeptide, and these sections may or may not be mutually exclusive from one another. In some embodiments, additional sections may optionally be included in the gRNA. The gRNA molecules utilized in the method described herein can vary in sequence length. The portion of the gRNA that is substantially complementary to the gRNA target, can also vary in length, and can be selected so that the gRNA targets the gRNA target sequences of different lengths. In some embodiments the portion of the gRNA targeted to a predetermined nucleic acid sequence, the predetermined nucleic acid sequence, or the entire gRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleic acids in length. In some embodiments, the gRNA target of the gRNA is about 15-25 nucleic acids in length. In some embodiments, the gRNA target of the gRNA is about 17-24 nucleic acids in length. In some embodiments, the gRNA target of the gRNA is about 20 nucleic acids in length. In some embodiments, the gRNA target of the gRNA is 20 nucleic acids in length. In some embodiments, the gRNA is a reverse complement of the gRNA target. In some embodiments, the gRNA comprises an RNA sequence having about 100% sequence identity to the reverse complement of the gRNA target. In some embodiments, the gRNA comprises an RNA sequence having at least about 99% sequence identity to the reverse complement of the gRNA target. In some embodiments, the gRNA comprises an RNA sequence having at least about 95% sequence identity to the reverse complement of the gRNA target. In some embodiments, the gRNA comprises an RNA sequence having at least about 90% sequence identity to the reverse complement of the gRNA target. In some embodiments, the gRNA comprises an RNA sequence having at least about 80% sequence identity to the reverse complement of the gRNA target.

The terms “nucleotide,” “polynucleotide,” “nucleic acid,” and “nucleic acid sequence” are used herein to refer to deoxyribonucleotides or ribonucleotides and polymers thereof in either single or double stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified versions thereof (e.g., degenerate codon substitutions) and complementary sequences and as well as the sequence explicitly indicated. Thus, the term nucleotide and the like is inclusive of the gRNAs that are described herein.

When a polynucleotide has a certain percentage of “sequence identity” to another polynucleotide, it means that the bases have the same percentage when aligned, and they are at the same relative positions when the two sequences are compared. Sequence identity can be determined in many different ways, for example sequences may be aligned using a variety of convenient methods and computer programs (e.g., BLAST, T-COFFEE, MUSCLE, MAFFT, etc.). The term “sequence identity” as used herein refers to the degree of sequence similarity on a nucleotide-by-nucleotide basis within a comparison window. Thus, “percentage of sequence identity” is calculated by comparing two optimally aligned sequences within a comparison window, the number of positions at which identical nucleic acid bases (e.g., A, T, C, G, or U) occur in the two sequences is determined to generate the number of matched positions, the number of matched positions is divided by the total number of positions in the comparison window (i.e., the window size), and the result is multiplied by 100 to give the percentage of sequence identity.

The terms “patient,” as used herein, refers to an individual organism, for example, a mammal, including, but not limited to, rodents, apes, humans, non-human primates, ungulates, felines, canines, bovines, sheep, mammalian farm animals, mammalian sport animals, and mammalian pets.

Currently, most studies directed towards a better understanding of the impact of specific TSC2 mutations on pathological processes have focused on inducing massive TSC2 disruption in cell lines, which allows the analysis of sporadic cases with severe phenotypes. In contrast, the induction of the most frequent type of SNP mutations in clinically relevant primary MSCs is disclosed herein, which is possible through the use of CRISPR-based base editing, offering unprecedented precision in modifying DNA. With its ability to precisely edit the genome, CRISPR-based base editing holds immense potential for advancing research in biology and medicine as well as for therapeutic applications.

Another critical determinant of successful CRISPR-Cas9 editing is the method of delivery to cells. Electroporation and lipofection are two prominent methods for delivering CRISPR-Cas9 components, each with distinct advantages and limitations. Electroporation uses electrical pulses to generate temporary pores in the cell membrane to facilitate transfer of components into a cell. Nucleofection, a type of electroporation, utilizes an electric field to facilitate the direct transfer of nucleic acids into the cell nucleus, often resulting in higher transfection efficiencies and greater success with hard-to-transfect cells or primary cells. Lipofectamine employs lipid-based reagents to form lipoplexes that fuse with the cell membranes and deliver CRISPR-Cas9 components into the cytoplasm. Lipofection is generally simple, cost-effective, and compatible with a wide range of cell types. Although reference to lipofection is disclosed herein, it should be appreciated by the person skilled in the art that nucleofection could be utilized instead.

Broadly, in a first aspect, a method for generating genomically engineered cells, the method comprising:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) culturing the pool comprising modified cells to produce a cultured pool; and
(d) selecting genomically engineered cells from the cultured pool wherein at least 35% of cells in the cultured pool comprise a mutation.

In some embodiments, the genomically engineered cells comprise at least one nucleotide point mutation.

In some embodiments, the mutation is in a Tuberous sclerosis complex 2 (TSC2) gene.

Accordingly, in some embodiments, the method for generating genomically engineered cells comprises:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) culturing the pool comprising modified cells to produce a cultured pool; and
(d) selecting genomically engineered cells from the cultured pool wherein at least 35% of cells in the cultured pool comprise a mutation in a Tuberous sclerosis complex 2 (TSC2) gene.

In some embodiments, the mutations in the TSC2 gene are in a Cyclin-B1 binding domain and GAP domain. In some embodiments, the mutation in the TSC2 gene comprises at one, any two, or all three substitutions selected from: a first cytosine to a first thymine at nucleic acid position 5024 (5024C>T); a guanine to an adenine at position 1832 (1832G>A); and a second cytosine to a second thymine at position 1864 (1864C>T). In some embodiments, the mutation in the TSC2 gene substitutes a first cytosine to a first thymine at nucleic acid position 5024 (5024C>T). In some embodiments, the mutation in the TSC2 gene substitutes a guanine to an adenine at position 1832 (1832G>A). In some embodiments, the mutation in the TSC2 gene substitutes a second cytosine to a second thymine at position 1864 (1864C>T). In some embodiments, the genomically engineered cells comprise one nucleotide point mutation. In some embodiments, the genomically engineered cells comprise two nucleotide point mutations. In some embodiments, the genomically engineered cells comprise three nucleotide point mutations.

In some embodiments, the sample cells comprise MSCs. In some embodiments, the MSCs are mammalian MSCs. In some embodiments, the MSCs are hMSCs. In some embodiments, the hMSCs are primary hMSCs. In some embodiments, the MSCs are patient-derived MSCs. In some embodiments, the hMSCs are patient-derived hMSCs. In some embodiments, a source of MSCs includes, but is not limited to, Wharton's jelly and cord blood.

In some embodiments, the base editing is performed using CRISPR, or a variation of CRISPR, as known to those skilled in the art. In some embodiments, the base editing is performed using the CRISPR-Cas system, which is a high-efficiency and cost-effective genome editing technology that can be widely applied to prokaryotes and eukaryotes. To date, based on the outstanding functional and evolutionary modularity of this system, CRISPR-Cas systems including six types (types I-VI) and two classes (class 1 and class 2) have been characterized. In class 2 of CRISPR-Cas systems, the CRISPR-Cas9 system is the most widely applied. For example, a traditional CRISPR-Cas9 system consists of a Cas9 nuclease and an engineered gRNA. The latter is responsible for guiding Cas9 to a target site which induces a double-stranded DNA break (DSB), and then the break site is repaired through endogenous pathways such as non-homologous end joining (NHEJ) and homology-directed repair (HDR). Details of the technical application of CRISPR-Cas systems and suitable guide RNA endonucleases are known to the skilled person and have been described in detail in the literature [see, e.g., Barrangou et al., 2016; Maeder et al., 2016; Cebrian-Serrano et al., 2017]. The present disclosure is not limited to the use of specific guide RNA endonucleases and therefore comprises the use of any given guide RNA endonucleases in the sense of the present invention suitable for use in the method described herein.

In some other embodiments, the base editing is performed using a base editor including, but not limited to, cytosine or adenine base editors (CBE or ABE). In still other embodiments, a prime editor is used to introduce genetic mutations into the genome of sample cells by using prime editing guide RNA (pegRNA).

In some embodiments, the Cas component is a ribonucleoprotein (RNP). In some embodiments, the Cas component is a DNA molecule encoding a Cas protein. In some embodiments, the Cas component is selected from one of Cas1B, Cas2, Cas3, Cas4, Cas5, Cash, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 polypeptide. In some embodiments, the Cas component is Cas9.

In some embodiments, the gRNA is designed by using a platform to target the mutation in the TSC2 gene, wherein the gRNA is selected based on a highest editing efficiency. In some embodiments, the disclosed editing method results in an on-target DNA base “editing efficiency” of at least about 35%, 40%, 50%, 60%, 70%, 80%, 85%, 90%, 95%, 98%, or 99% at the target nucleotide. In some embodiments, a “high editing efficiency” of at least 85% is achieved. In some embodiments, the gRNA is designed by using a platform to target the mutation in the TSC2 gene, wherein the gRNA is selected based on a lowest off-target efficiency. In some embodiments, the disclosed editing method results in a low actual or average “off-target editing efficiency” or “off target efficiency” of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In some embodiments, the gRNA is designed by using a platform to target the mutation in the TSC2 gene, wherein the gRNA is selected based on both a highest editing efficiency and a lowest off-target efficiency. In some embodiments, the platform used to target the mutation includes, but is not limited to, the TrueDesign Invitrogen platform, Genscript gRNA Design Tool, or the Benchling CRISPR Guide RNA Design Tool). In some embodiments, the gRNA is a guide RNA directed to a gRNA target. In some embodiments, the gRNA is a guide RNA directed to a location in a TSC2 gene. In some embodiments, the gRNA target is located in a TSC2 gene. In some embodiments, the gRNA target comprises a sequence with at least 90% sequence identity to the sequence of SEQ ID NO: 1, SEQ ID NO: 2, or SEQ ID NO: 3. In some embodiments, the gRNA target comprises a sequence with at least 95% sequence identity to the sequence of SEQ ID NO: 1, SEQ ID NO: 2, or SEQ ID NO: 3. In some embodiments, the gRNA target comprises the sequence set forth in any one of SEQ ID NO: 1, SEQ ID NO: 2, or SEQ ID NO: 3.

In some embodiments, the ratio of Cas component to gRNA is in a range from about 1.5:0.5 to about 2.5:0.75.

In some embodiments, the HDR template, or donor DNA, comprises a sequence with at least 90% sequence identity to the sequence of SEQ ID NO: 4, SEQ ID NO: 5, or SEQ ID NO: 6. In some embodiments, the HDR template comprises a sequence with at least 95% sequence identity to the sequence of SEQ ID NO: 4, SEQ ID NO: 5, or SEQ ID NO: 6. In some embodiments, the HDR template comprises the sequence set forth in any one of SEQ ID NO: 4, SEQ ID NO: 5, or SEQ ID NO: 6.

In some embodiments, cells are cultured in one of any suitable culture media. Different culture media having different ranges of pH, glucose concentration, growth factors, amino acids, and other supplements can be used for different cell types or for different applications. In some embodiments, custom cell culture media or commercially available cell culture media such as Dulbecco's Modified Eagle Medium, Minimum Essential Medium, RPMI medium, HA medium, HAT medium, RoosterBasal-MSCs, RoosterNourish™-MSC (SKU/Catalog No: K82003) and/or RoosterBooster-MSC-XF (RoosterBio Inc.), or other media available from Life Technologies, RoosterBio® or other commercial sources can be used. In some embodiments, cell culture media include serum (e.g., fetal bovine serum, bovine calf serum, equine serum, porcine serum, or other serum). In some embodiments, cell culture media are serum-free. In some embodiments, cell culture media include human platelet lysate (hPL). In some embodiments, cell culture media include one or more antibiotics (e.g., actinomycin D, ampicillin, carbenicillin, cefotaxime, fosmidomycin, gentamycin, kanamycin, neomycin, penicillin, penicillin streptomycin, polymyxin B, streptomycin, tetracycline, or any other suitable antibiotic or any combination of two or more thereof). In some embodiments, cell culture media include one or more salts (e.g., balanced salts, calcium chloride, sodium chloride, potassium chloride, magnesium chloride, etc.). In some embodiments, cell culture media include sodium bicarbonate. In some embodiments, cell culture media include one or more buffers (e.g., HEPES or other suitable buffer). In some embodiments, one or more supplements are included. Non-limiting examples of supplements include reducing agents (e.g., 2-mercaptoethanol), amino acids, cholesterol supplements, vitamins, transferrin, surfactants (e.g., non-ionic surfactants), CHO supplements, primary cell supplements, yeast solutions, or any combination of two or more thereof. In some embodiments, one or more growth or differentiation factors are added to cell culture media. Growth or differentiation factors (e.g., WNT-family proteins, BMP-family proteins, IGF-family proteins, etc.) can be added individually or in combination, e.g., as a differentiation cocktail comprising different factors that bring about differentiation toward a particular lineage. Growth or differentiation factors and other aspects of a liquid media can be added using automated liquid handlers integrated within the incubators. Methods of culturing cells are well known in the art.

In some embodiments, using the method described herein, at least 40% of cells in the pool of genomically engineered cells comprises a mutation in the TSC2 gene. In some embodiments, using the method described herein, at least 50% of cells in the pool of genomically engineered cells comprises a mutation in the TSC2 gene. In some embodiments, using the method described herein, at least 60% of cells in the pool of genomically engineered cells comprises a mutation in the TSC2 gene. In some embodiments, using the method described herein, at least 70% of cells in the pool of genomically engineered cells comprises a mutation in the TSC2 gene. In some embodiments, using the method described herein, at least 80% of cells in the pool of genomically engineered cells comprises a mutation in the TSC2 gene.

Suitable methods for delivering the machinery required to induce genetic modifications (also referred to as “transformation”) include, but are not limited to, viral or phage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, direct microinjection, nanoparticle-mediated nucleic acid delivery, and the like. The choice of delivery method of genetic modification generally depends on the type of the cell to be transformed and the circumstances under which the transformation occurs (e.g., in vitro, ex vivo, or in vivo). In some embodiments, the delivery method for genetic modification comprises lipofection. In some other embodiments, the delivery method for genetic modification comprises electroporation. In some other embodiments, the delivery method for genetic modification comprises nucleofection. In some embodiments, the delivery method for genetic modification comprises the use of a Lonza 4D-Nucleofector™ or Neon NxT Elextroporation System. In some embodiments, the delivery method for genetic modification comprises the use of Lipofectamine. In some embodiments, the delivery method for genetic modification comprises the use of Lipofectamine CRISPRMAX. In some embodiments, the delivery method for genetic modification comprises a ratio of Cas component to gRNA to Lipofectamine CRISPRMax to a Cas9 Plus reagent. In some embodiments, the ratio of Cas component to gRNA to Lipofectamine CRISPRMax to a Cas9 Plus reagent is 500-1500 ng:500-1000 ng:1-10 μL:1-5 μL. In some embodiments, the ratio of Cas component to gRNA to Lipofectamine CRISPRMax to a Cas9 Plus reagent is 750-1250 ng:600-900 ng:3-9 μL:2-4 μL. In some embodiments, the ratio of Cas component to gRNA to Lipofectamine CRISPRMax to a Cas9 Plus reagent is 900-1100 ng:700-800 ng:4-8 μL:2.5-3.5 μL. In some embodiments, the ratio of Cas component to gRNA to Lipofectamine CRISPRMax to a Cas9 Plus reagent is 1 ug:750 ng:6 μL:3 μL.

Accordingly, in another embodiment, a method for generating genomically engineered cells comprises:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) electroporating the Cas component, the gRNA, and HDR template in the pool of sample cells using an electroporation agent;
(d) culturing the pool comprising modified cells to produce a cultured pool; and
(e) selecting genomically engineered cells from the cultured pool wherein at least 35% of cells in the cultured pool comprise a mutation.

In another embodiment, the method for generating genomically engineered cells comprises:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) electroporating the Cas component, the gRNA, and HDR template in the pool of sample cells using an electroporation agent;
(d) culturing the pool comprising modified cells to produce a cultured pool; and
(e) selecting genomically engineered cells from the cultured pool wherein at least 35% of cells in the cultured pool comprise a mutation in a Tuberous sclerosis complex 2 (TSC2) gene.

In some embodiments, the electroporation agent comprises nucleofector.

In another embodiment, a method for generating genomically engineered cells comprises:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) transfecting the Cas component, the gRNA, and HDR template in the pool of sample cells using a lipid-based transfection agent;
(d) culturing the pool comprising modified cells to produce a cultured pool; and
(e) selecting genomically engineered cells from the cultured pool wherein at least 35% of cells in the cultured pool comprise a mutation.

In another embodiment, the method for generating genomically engineered cells comprises:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) transfecting the Cas component, the gRNA, and HDR template in the pool of sample cells using a lipid-based transfection agent;
(d) culturing the pool comprising modified cells to produce a cultured pool; and
(e) selecting genomically engineered cells from the cultured pool wherein at least 35% of cells in the cultured pool comprise a mutation in a Tuberous sclerosis complex 2 (TSC2) gene.

In some embodiments, the lipid-based transfection agent comprises a lipofectamine agent. In some embodiments, the lipofectamine agent comprises Lipofectamine 2000. In some embodiments, the lipofectamine agent comprises Lipofectamine 3000. In some embodiments, the lipofectamine agent comprises Lipofectamine CRISPRMAX.

In some embodiments of the methods described herein, the genomically engineered cells are assayed for cell viability. In some embodiments, at least 60% of cells in the pool of genomically engineered cells are viable for fourteen days following transformation (e.g., transfection). In some embodiments, at least 70% of cells in the pool of genomically engineered cells are viable for fourteen days following transformation (e.g., transfection). In some embodiments, at least 75% of cells in the pool of genomically engineered cells are viable for fourteen days following transformation (e.g., transfection). In some embodiments, at least 80% of cells in the pool of genomically engineered cells are viable for fourteen days following transformation (e.g., transfection).

In some other embodiments, a method of inducing a single nucleotide point mutation into a mesenchymal stem cell is described, the method comprising:
introducing into the mesenchymal stem cell a Cas component, a guide RNA (gRNA) targeting a TSC2 gene, and a homology directed repair (HDR) template comprising a single nucleotide point mutation and transforming same to generate a genetically engineered mesenchymal stem cell comprising a mutation in a Tuberous sclerosis complex 2 (TSC2) gene.

It should be appreciated by the person skilled in the art that the methods described herein can further comprise isolating a genomically engineered cell comprising the mutation in the TSC2 gene, and optionally culturing the genomically engineered cell to generate a mutated TSC2 cell line, as understood by the person skilled in the art.

In another aspect, a CRISPR composition is described, said composition comprising:
(a) a Cas9 protein;
(b) a guide RNA (gRNA) directed to a gRNA target, wherein the gRNA target sequence comprises a sequence with at least 90% sequence identity to the sequence of SEQ ID NO: 1, SEQ ID NO: 2, or SEQ ID NO: 3; and
(c) a homology directed repair template (HDR), wherein the HDR template comprises a sequence with at least 90% sequence identity to the sequence of SEQ ID NO: 4, SEQ ID NO: 5; or SEQ ID NO: 6.

Advantageously, the high editing efficiency of some TSC2 mutations using the methods described herein also allows for the reversal of mutations. In some embodiments, to reverse single point mutations, a new gRNA sequence and a new HDR template is obtained and used. Reversing the mutation, as described herein, would make this method a therapeutic agent, which can provide patients carrying the mutation, with a curative therapy.

In still another aspect, a method for correcting or reversing a pathogenic mutation in a TSC2 gene is described, the method comprising:
(a) providing a pathogenic cell comprising the pathogenic mutation at the TSC2 gene;
(b) introducing into the pathogenic cell a Cas component, a guide RNA (gRNA) directed to a gRNA target, and a corrective homology directed repair (HDR) template to produce a corrected cell,
wherein the corrective HDR template comprises a nonpathogenic sequence of the TSC2 gene.
   In some embodiments, the pathogenic mutation is a single nucleotide point mutation.

In some embodiments of this aspect, a method for treating a subject with a pathogenic mutation in a TSC2 gene is described, the method comprising:
providing the subject comprising the pathogenic mutation at the TSC2 gene a therapeutically effective dose of:
(a) a Cas component,
(b) a guide RNA (gRNA) directed to a gRNA target, and
(c) a corrective homology directed repair (HDR) template comprising a nonpathogenic sequence of the TSC2 gene.
   In some embodiments, the pathogenic mutation is a single nucleotide point mutation.

In some embodiments of this aspect, a therapeutic agent for treating a disease caused by a mutation in a pathogenic TSC2 gene is described, the therapeutic agent comprising:
(a) a Cas component;
(b) a guide RNA (gRNA) targeted to the pathogenic TSC2 gene; and
(c) a corrective homology directed repair (HDR) template encoding a nonpathogenic TSC2 gene.
   In some embodiments, the pathogenic mutation is a single nucleotide point mutation. In some embodiments, the disease is cancer. In some embodiments, the disease is tuberous sclerosis complex.

Successful CRISPR base editing of TSC2 in mesenchymal stem cells (MSCs) represents a significant advancement in gene editing and cancer research. Leveraging the precision of CRISPR-based base editing of TSC2 enables the simulation of TSC2-derived pathologies, including cancer diseases and tuberous sclerosis complex, and holds transformative potential for treating these conditions. Moreover, lipofectamine CRISPRMax is a safe option for delivering CRISPR Cas9 components into hMSCs and facilitating high-throughput editing scores. Notably, one off-target mutation was observed within the PAM site, which was not predicted by current gRNA design tools. Therefore, in silico tools should only be used as a guide and verified by in vitro experiments. In this context, hMSCs, e.g., derived from Wharton's jelly and cord blood, can be excellent non-invasive sources for verifying the precision and efficacy of genome editing, especially at the neonatal stage when sampling tissue from patients can be challenging. Although Sanger sequencing serves as a reliable initial tool, NGS provides the necessary depth and precision for a thorough assessment of CRISPR-based base-editing outcomes. Consequently, both methods are often employed in tandem to ensure the accurate and comprehensive validation of genome editing experiments. Overall, the high editing efficiency of some TSC2 mutations allows for the induction and reversal of mutations in widely available primary hMSCs without requiring time- and resource-consuming derivation of cell lines that are frequently distinct from their primary counterparts.

### EXAMPLES

### Methods

### Cell Types and Cell Cultures

HEK 293 cells, acquired from ATCC™, were cultured in Dulbecco's Modified Eagle Medium (DMEM) supplemented with 10% fetal bovine serum (FBS) (Gibco, Waltham, MA, USA). All cell types were passaged every 2-3 days, maintained below 80% confluence, and cultured in 75-cm² cell culture flasks at 37° C. with 5% CO₂. Human MSCs (hMSCs) isolated from bone marrow under xeno-free conditions (RoosterVial-hBM-1M-XF) were sourced from healthy adult donors of both sexes, aged 18-30 years, through RoosterBio, Inc., USA. The cells were thawed and cultured in “standard medium,” RoosterNourish-MSC-XF, a combination of RoosterBasal-MSCs (Cat. No. SU-005), and RoosterBooster-MSC-XF (RoosterBio Inc.). The cells were maintained in a humidified environment at 37° C. and 5% CO₂ in 75-cm² cell culture flasks for one passage before electroporation or transfection, with the culture medium changed every two days. Mycoplasma testing was performed regularly before each experiment to ensure that the cells were not contaminated. For subsequent experiments, HEK293 cells and hMSCs (from the second passage) were treated with trypsin-EDTA (0.05%) and phenol red, and then transferred to 24-well plates at a density of 2.5×10⁴ cells per well, or 96-well plates at a density of 1×10⁴ cells per well. All cell groups had a high viability above 90% prior to the experiments.

### Systematic Design of Experiments for Optimized CRISPR/Cas9 Genome Editing.

The feasibility of inducing the most frequently reported SNP mutations in TSC2 was determined to maximize the clinical relevance of the results. Thorough research was conducted utilizing various genetic databases. In the initial search for the most frequent TSC2 SNP mutation in NCBI ClinVar, P1 TSC2.1864C>T (p.Arg622Trp) was reported to be the most frequent. Therefore, this mutation was selected as the first target to demonstrate its ability to induce TSC2 SNP mutations in the human genome. During the secondary search for correlations between cancer and TSC2 SNP mutations, the Leiden Open Variation Database (LOVD) and Human Gene Mutation Database (HGMD) presented P2 TSC2.1832 G>A (p.Arg611Glu) and P3 TSC2.5024 C>T (p.Pro1675Leu) as the most frequent and highly correlated with cancerous tumors, whereas P1 correlated only with mild TSC presentation. Therefore, further experiments and downstream analyses focused on P2 and P3 SNP mutations. Nonetheless, all three mutations are located in different TSC2 domains, which provides a broader representation of mutation locations and increases the generalizability of the findings. Using the platform Invitrogen TrueDesign (ThermoFisher), several guide RNA (gRNA) target sequences were identified to target each mutation, as shown in Table 1. The gRNAs with the highest predicted editing efficiency and the lowest off-target yield corresponding to the identified gRNA targets were selected for further experiments. To increase editing precision, single-stranded donor DNA (ssDonor DNA) molecules were designed to serve as homology-directed repair (HDR) templates (see, Table 1). Potential off-target sites were predicted by in silico analysis using the TrueDesign Genome Editor™ and Cas-OFFinder 2. To ensure the highest compatibility, TrueCut™ CRISPR/Cas9 Ribonucleoprotein (RNP) V2 from Invitrogen Thermo Fisher was chosen for base editing. Editing of P1 TSC2.1864C>T (p.Arg622Trp) was used to prove the feasibility of performing SNP base editing of TSC2 in HEK293 and mesenchymal cells. Therefore, genomic cleavage detection and assessment of CRISPR cytotoxicity were not performed.

TABLE 1
Guide RNA and guide RNA target sequences

| Target point | gRNA Target | |
|---|---|---|
| mutation | (5′→3′) | Donor DNA (5′→3′) |
| TSC2.1832 G > A | TGCCAATCGC | ACAAGCACAGCTACACCCTGCCAATCGCGAG |
| (p.Arg611Glu) | GAGCAGCATC | CAGCATTCAGCTGCAGGTATGGTGGCTGGGGT |
| | (SEQ ID NO: 1) | TGCGCAGCCAGT (SEQ ID NO: 4) |
| TSC2.5024 C > T | AGGTTGCACT | GGGCCAGTTCAACTTTGTCCACGTGATCGTCA |
| (p.Pro1675Leu) | CGTAGTCCAG | CCCTGCTGGATTACGAGTGCAACCTGGTGTCC |
| | (SEQ ID NO: 2) | CTGCAGTGCAG (SEQ ID NO: 5) |
| TSC2.1864 C > T | CACTGCACCG | CTTCCTGACAGGCCTTTGACTTCCTGTTGCTGC |
| (p.Arg622Trp) | CCTGGGC (SEQ | TGTGGGCCGATTCACTGCACCGCCTGGGCCTG |
| | ID NO: 3) | CCCAACAAGGATGGA (SEQ ID NO: 6) |
| Control | AAAUGUGAGA | N/A |
| (nonfunctional | UCAGAGUAAU | |
| gRNA) | (SEQ ID NO: 7) | |

### Delivery of CRISPR-Cas9 Using Electroporation

The editing efficiency and cytotoxicity of the two electroporation systems in the two cell types were compared. The Lonza 4D-Nucleofector™ and Neon NxT Electroporation systems are both designed for the efficient electroporation of various primary cells, including MSCs. Both systems use nucleofection modules, which would allow for precise control over the electroporation parameters, explicitly tailored to hMSCs, ensuring minimal cellular stress and high post-transfection survival rates. CRISPR-Cas9 pre-optimized protocols with HDR repair for both the devices were used. Cells were harvested and mixed with CRISPR RNP, gRNA, ssDonorDNA, and nucleofector solution. In the first run, base editing was performed in HEK293 cells to ensure the functionality of the CRISPR-Cas9 system. After achieving satisfactory editing efficiency, base editing was performed to separately induce the three selected mutations in hMSCs. For the Lonza 4D-Nucleofector, single Nucleocuvette™ (100 μL) and 16-well Nucleocuvette™ Strips (20 μL) were used. Electroporation conditions (FF-104) were used to achieve high efficiency. For the Neon NxT system, 10 μL of nucleofection solution, containing 50,000 cells per reaction, was loaded into the nucleofection pipette. Several electroporation conditions were tested: [1000 V, 40 ms, one pulse], [1200 V, 30 ms, two pulses], and [1300 V, 40 ms, two pulses]. After the electrical pulse, the cells were transferred to prewarmed culture media and incubated under standard culture conditions to allow for recovery.

### Optimization of Lipofectamine-Based Transfection Process

Transfection reagents were screened using two different lipofectamine agents to deliver CleanCap® Enhanced Green Fluorescent Protein mRNA (Trilink) into hMSC. Prior to transfection, cell populations were seeded in 24-well plates. Transfection was performed on hMSC seeded in 48-well plates, reaching a confluence of 60% on the day of transfection. The amounts of eGFP-mRNA, Lipofectamine 2000, and Lipofectamine CRISPRMAX were used as recommended in the manufacturer's protocol (Human Mesenchymal Stem Cells Lipofectamine® 2000, Lipofectamine™ CRISPRMAX™ Transfection Reagent Pub. No. MAN0014545 Rev. B.0). Several volume ratios of Cas9 Plus reagent to Lipofectamine CRISPRMAX were used, starting with 2.5/1.5 μL, 5/2.5, and 6/3 μL per 500 μL cell medium per well, and the eGFP mRNA was increased to 1.5, 2 and 2.5 μg per reaction, respectively. Lipofectamine 2000 was used at 3 μL per 500 μL per well with 2 μg of eGFP-mRNA. Upon 24 h post-transfection incubation, the Spark® Cyto Multimode Microplate reader (Tecan) was used to compare the GFP signal expression between hMSCs transfected with Lipofectamine 2000 and various concentrations of Lipofectamine CRISPRMAX. Cell viability was analyzed using the Cell Counting Kit-8 (CCK-8) (Dojindo Molecular Technologies Inc Cytotoxicity LDH Assay Kit-WST).

### Delivery of Cas9 Components Through Transfection in a 24-Well Plate Using CRISPRMAX

One day prior to transfection, adherent cells were plated in 24-well plates at approximately 50,000 cells per well in 500 μL of growth medium so that the cells reached 50-70% confluence at the time of transfection. In the first run of transfection, 25 μL of Opti-MEM medium was added to a 1.5 mL sterile Eppendorf tube, followed by 500 ng TrueCut Cas9 V2 and 500 ng gRNA. Upon mixing by brief vortexing, 2 μL Cas9 Plus reagent was added to the solution containing Cas9 protein and gRNA. After brief vertexing, the mixture was incubated at 25° C. for 5 min to allow for the formation of Cas9 RNPs. For the co-delivery of donor DNA, 500 ng of single-stranded DNA oligonucleotides was added to the Cas9 RNPs. Meanwhile, 25 μL of Opti-MEM medium was added to a separate sterile Eppendorf tube, followed by 3 μL of Lipofectamine CRISPRMAX. After brief verification, the Lipofectamine CRISPRMAX solution was incubated at 25° C. for approximately 5 min. After incubation, Cas9 RNPs were added to the Lipofectamine CRISPRMAX solution. Upon mixing, the sample was incubated at 25° C. for 10-15 min to form Cas9 RNPs and Lipofectamine CRISPRMAX complexes, and then added to the cells. For the second run of transfection, 1 μg GeneArt Platinum Cas9 nuclease, 750 ng gRNA, and 6 μL Cas9 Plus reagent were used to prepare the Cas9 RNPs, and the amount of Lipofectamine CRISPRMAX reagent was accordingly increased to 3 μL. A 50 μl transfection mixture was added to 450 μL of cell medium to reach a final volume of 500 μL per well. The negative control was transfected with a non-targeting sgRNA (5′-AAAUGUGAGAUCAGAGUAAU-3′ (SEQ ID NO: 7). At 48-72 h post-transfection, the cells were harvested for genome modification efficiency analysis using the GeneArt Genomic Cleavage Detection kit.

### Genomic Cleavage Assay

The GeneArt Genomic Cleavage Detection Kit (ThermoFisher) enables the detection of the cleavage activity of CRISPR-Cas9 within the target genomic DNA. By using a combination of primers and a proprietary assay, indels (insertions or deletions) resulting from CRISPR-induced double-strand breaks can be identified. A kit was used for validating CRISPR-Cas9 DNA cleavage in the transfected hMSCs with mutation P2 TSC2.1832 G>A (p.Arg611Glu), mutation P3 TSC2.5024 C>T (p.Pro1675Leu), and their negative control cells. The cleavage detection experiment was performed as described in the manufacturer's manual. At 72 h post-transfection, Cells were lysed with 50 μL cell lysis buffer per 24-well. Three pairs of Primers were designed for each point mutation using the Invitrogen TrueDesign platform (see, Table 2). The polymerase chain reaction (PCR) program was set at 95° C. for 3 min for one cycle, then at 95° C. for 30 s, 55° C. for 30 s, and 72° C. for 30 s for a total of 40 cycles. The final extension was set at 72° C. for 5 min. The resulting PCR product (3μ) was mixed with 1 μL of 10× Detection Reaction Buffer and 5 μL water and then subjected to denaturation and re-annealing at 95° C. for 5 min, 4° C. for 5 min, 37° C. for 5 min, and then 4° C. for 5 min. Finally, 1 μL 10× detection enzyme was added to the sample and then incubated at 37° C. for 1 h. The digested product was analyzed with a 2% gel 1× agarose. The cleavage was visualized using the imaging system Gel Doc XR+ (Biorad).

TABLE 2
Genomic Cleavage Detection Primers

| Target point | Forward primers | Primer | Reverse primers | Primer |
|---|---|---|---|---|
| mutation | (5′→3′) | Tm | (5′→3′) | Tm |
| TSC2.1832 | TCTGCAGACCAAGCTG | 59.0 | GTCCTTTTCTCTGCCCC | 58.8 |
| G > A | TACA (SEQ ID NO: 8) | | AAC (SEQ ID NO: 9) | |
| (p.Arg611Glu) | TTTGCTGCTGTGGAGA | 58.9 | CTCTGCAGCTTCCAGG | 59.7 |
| | GAGA (SEQ ID NO: 10) | | AAC (SEQ ID NO: 11) | |
| | CTGGGTTTGAAGGTCG | 59.1 | ACAGATGTGTGGACTG | 60.0 |
| | TGTG (SEQ ID NO: 12) | | CAGG (SEQ ID NO: 13) | |
| TSC2.5024 | TAGAGGTGTCTTGCCT | 59.0 | ATGTCTGGGGAGACTT | 59.0 |
| C > T | GTGG (SEQ ID NO: 14) | | GGTG (SEQ ID NO: 15) | |
| (p.Pro1675Leu) | TAGCCGAGATCAGCCT | 59.0 | TTGCGGTCAGACACGA | 59.7 |
| | TCAG (SEQ ID NO: 16) | | TCTT (SEQ ID NO: 17) | |
| | AGCTGACAGGTGTCTA | 59.1 | ACTCACATTTGCGTGC | 59.0 |
| | GCAG (SEQ ID NO: 18) | | AGG (SEQ ID NO: 19) | |
| TSC2.1864 | CAGAGCCTGTGTCTGT | 58.8 | CCGCTGGTCTTCTTCTC | 59.1 |
| C > T | GTTG (SEQ ID NO: 20) | | AGA (SEQ ID NO: 21) | |
| (p.Arg622Trp) | CCTTTTCTGAGTGCCTG | 58.8 | GCAGGAACGGAACAG | 59.1 |
| | TGG (SEQ ID NO: 22) | | ACTTG (SEQ ID NO: 23) | |
| | GATGTGGGTGTGTGCA | 59.5 | GGACATCCCTCAGACA | 59.5 |
| | CATC (SEQ ID NO: 24) | | TGCA (SEQ ID NO: 25) | |

### Assessment of Cytotoxicity

This study aimed to evaluate the safety of CRISPR-Cas9 base editing in hMSCs. Using the Cell Counting Kit-8 (CCK-8) and Trypan blue exclusion assays, the viability of hMSCs with SNP edits P2 TSC2.1832 G>A (p.Arg611Glu) and P3 TSC2.5024 C>T (p.Pro1675Leu) and their negative controls were monitored. As HEK293 cells and hMSCs with SNP edit P1 TSC2.1864C>T (p.Arg622Trp) were only used to prove the functionality of the CRISPR-Cas9 system, no cell behavior was observed after the base-editing experiments. Cell samples were prepared in 96-well plates at approximately 10,000 cells/well. For nucleofected cells, CCK8 assays were performed starting on days 1, 3, and 7 after electroporation. Because the transfection protocols recommend a minimum incubation time of 48 to 72 h, CCK8 was performed on days 3, 7, and 14 after the experiment. Cells were allowed to adhere before treatment with CCK-8 reagent, typically at a volume of 10 μL per 100 μL of culture medium. After incubating the plates at 37° C. for 1-4 hours, the absorbance of each well was measured at 450 nm using a Spark® Cyto Multimode Microplate reader (Tecan). Cells were harvested at the same intervals as in the CCK8 assay for cell viability detection. Ten microliters of cell suspension were mixed with 10 μL of Trypan Blue solution. After incubation for a few minutes, the mixture was loaded onto a counting chamber and cell viability was measured using Cell Profiler Automated Cell Counter 2000 Nexcelom®.

The resulting CCK8 absorbance of the two transfected hMSCs and control groups were compared using one-way analysis of variance (ANOVA). The same approach was used for electroporated cells and control hMSCs. Differences between the groups were considered significant at P<0.05. Statistical analyses were performed using GraphPad Prism 10.0 (GraphPad Software, Inc., San Diego, CA, USA).

Analysis of Mutation Frequencies and Off-Target Effects within the Protospacer Adjacent Motif (PAM) Site

To perform Sanger sequencing after CRISPR editing, genomic DNA was extracted using the DNeasy Blood & Tissue Kit (Qiagen Pty Ltd., Hilden, Germany). Approximately 1 million cells per sample were harvested from the DNA, according to the manufacturer's protocol (DNeasy® Blood & Tissue Kit, cat. No. 69506). DNA concentrations were measured using either a NanoDrop ND-1000 UV/VIS Spectrophotometer or Qubit 4 Fluorometer (ThermoFisher™). Primers were designed to generate amplicons with sizes between 200 and 500 base pairs that included the SNP editing and PAM sites of each mutation, as recited in Table 3. Polymerase chain reaction (PCR) amplification was performed using Phusion High-Fidelity DNA Polymerase (ThermoScientific) in accordance with the manufacturer's protocol (Pub. No. MAN0012393) using a MiniAmp Thermal Cycler (Thermofisher™). After amplification, PCR products were purified, and Sanger sequencing was performed using Genewiz sequencing services from Azenta Life Sciences.

TABLE 3
Sanger sequencing primers. The forward primers include a 5′ M13 sequence
TGTAAAACGACGGCCAGT (SEQ ID NO: 32) for downstream sequencing

| Target point | Forward primers | Primer | Reverse primers | Primer |
|---|---|---|---|---|
| mutation | (5′→3′) | Tm | (5′→3′) | Tm |
| TSC2.1832 | CCACTACAAGCACAGCTA | 61.7 | GCCAACATCTATAGC | 61.7 |
| G > A | CA (SEQ ID NO: 26) | | GCAAAC (SEQ ID NO: | |
| (p.Arg611Glu) | | | 27) | |
| TSC2.5024 | ATTAGAGGTGTCTTGCCT | 61.8 | CCAGGTTGCACTCGTA | 61.8 |
| C > T | GTG (SEQ ID NO: 28) | | GTC (SEQ ID NO: 29) | |
| (p.Pro1675Leu) | | | | |
| TSC2.1864 | GGACACCAGGCTCTGTGA | 60.2 | TCAGAGCCTCTCTCTG | 59.8 |
| C > T | G (SEQ ID NO: 30) | | GCTC (SEQ ID NO: 31) | |
| (p.Arg622Trp) | | | | |

Finally, a comprehensive analysis of the sequencing data was performed using the bioinformatics software Snapgene and the ICE CRISPR Analysis Tool (Synthego). The obtained sequences were compared to the reference sequences to identify the precise nature of CRISPR-induced edits. Insertions, deletions, or other alterations introduced by CRISPR were also searched for and any potential off-target effects assessed within the generated amplicons.

### Results

### Efficiency of Lipofectamine CRISPRMAX

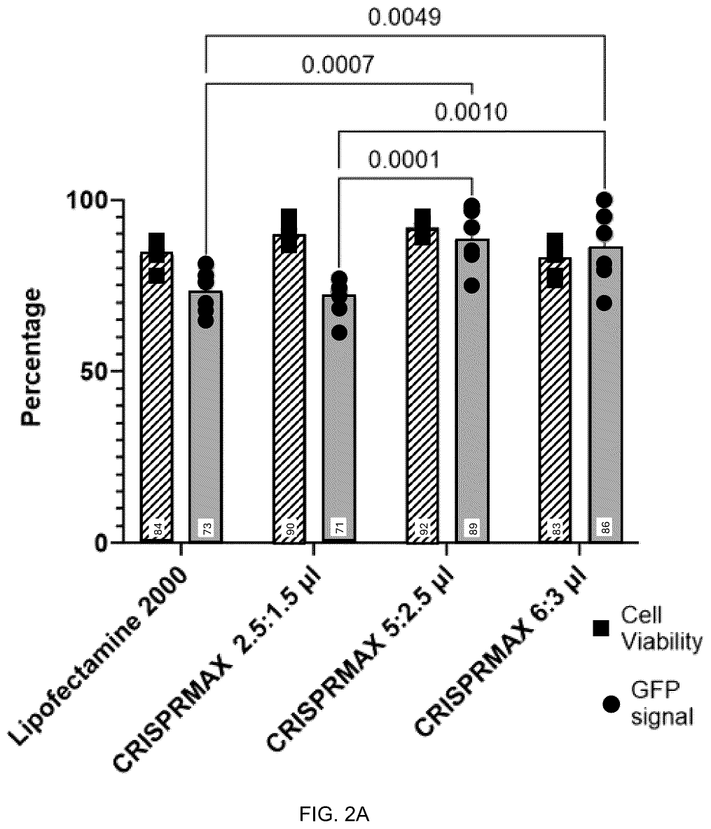

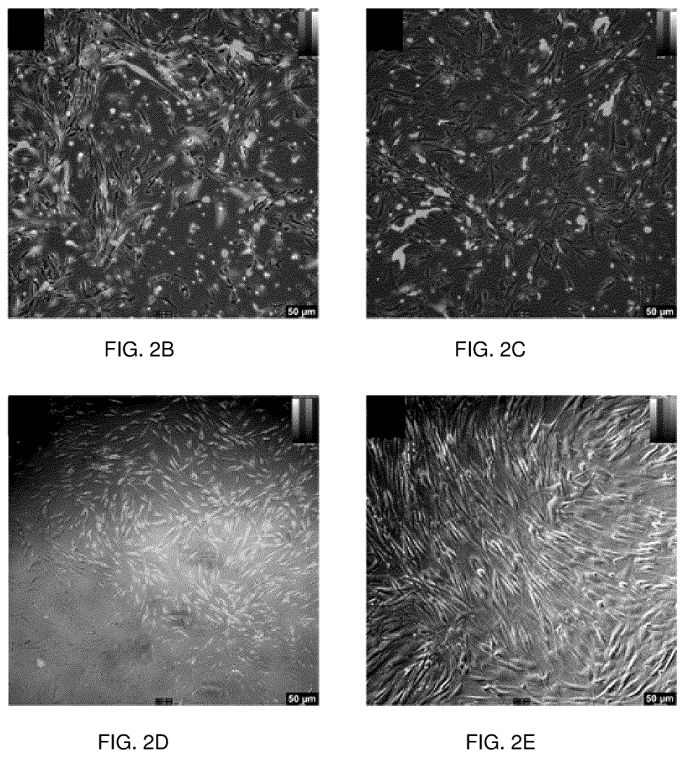

hMSCs transfected with eGFP using Lipofectamine 2000 showed that the fluorescence signal intensity reached a maximum of 73% at 24 h and 48 h after transfection. Using Lipofectamine CRISPRMAX, a similar fluorescent signal of 71% was reached at 2.5:1.5 μL Cas9 reagent to lipofectamine CRISPRMAX ratio. After an increase in the ratio to 5:2.5 μL, the fluorescence increased to 87%; no significant increase was observed at 86% after the increase in the ratio to 6/3 μL (see, FIGS. 2C-2E). The eGFP-mRNA mass used was at 1.5 μg for the first reaction, 2 μg, and 2.5 μg for the first, second, and third reactions, respectively. Cell viability remained stable at 80-92% in all transfected cells on day 3 post-transfection (FIG. 2A). No significant differences in cell viability were found between all cell groups. Therefore, 5:2.5 μL Cas9 reagent to lipofectamine CRISPRMAX ratio was determined to be optimal and was selected for CRISPR/Cas9 experiments.

### Predicted and Obtained Gene Editing Efficiency and Off-Targets

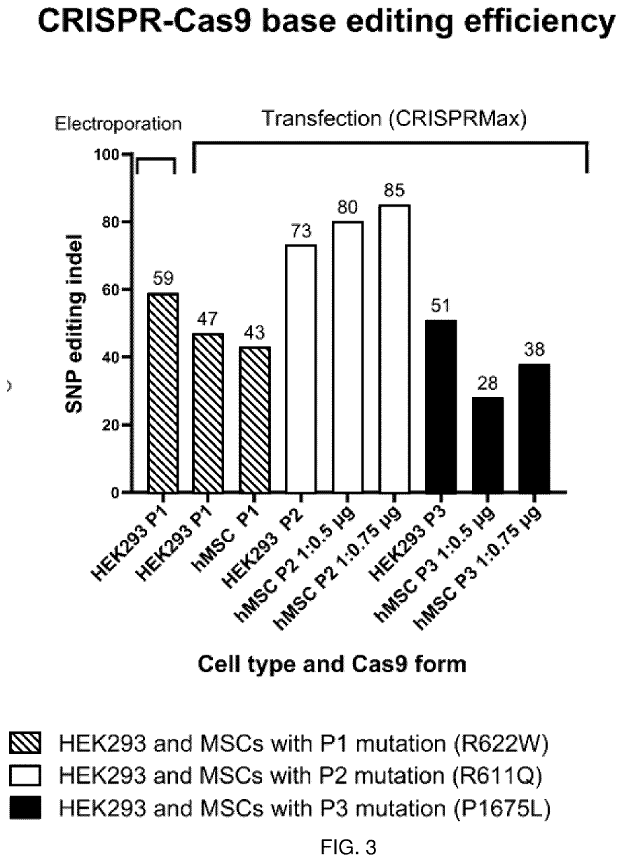

Using Lonza 4D electroporation on HEK293 cells, 59% editing efficiency of P1 TSC2.1864C>T (p.Arg622Trp) was achieved (see, FIG. 3). Despite multiple attempts using the two devices Lonza 4D and Neon NxT, subsequent electroporation of hMSCs displayed severe loss of viability, precluding DNA sequence analysis.

Lipofection using CRISPRMax with a Cas9 to gRNA ratio of 1500:500 ng for P1 resulted in a successful editing score of 47% in HEK293 cells and 43% in hMSCs (FIG. 3). The editing efficiency of the point mutation P2 was 73% in HEK293 cells, and 80% in hMSCs using a Cas9 to gRNA ratio of 1500:500 ng. After an increase of the Cas9 to 2500 and the gRNA amount to 750 ng, the editing efficiency increased to 85% (FIG. 3). Lastly, the point mutation P3 reached an indel of 51% in HEK293 cells and 28% in hMSCs. Increasing the Cas9 to 2500 ng and the gRNA concentration to 750 ng per reaction in hMSCs resulted in an editing efficiency of 38% (FIG. 3). Meanwhile, in silico predicted efficiencies of the designed gRNAs were 86.49% for P1, 98.57% for P2, and 87% for P3.

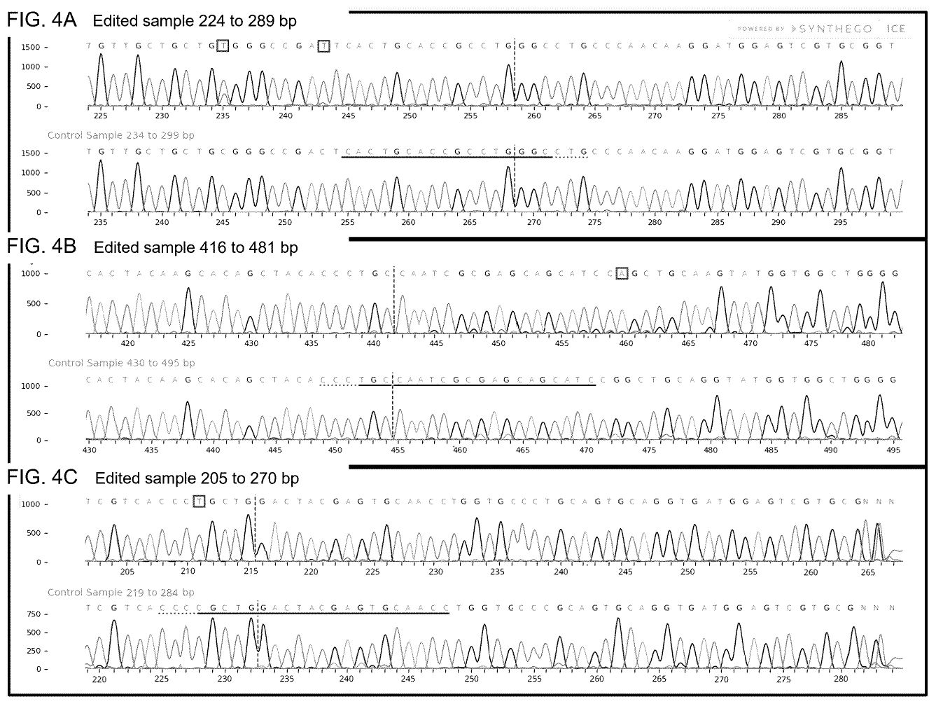

While designing the gRNA sequence targeting, potential off-target sites were searched for in silico; the gRNAs with the least off-targets for both mutations and no off-target mutations within the PAM site were chosen. Unexpectedly, the results showed an undesired edit within the PAM site in P1 at position 1839 (G>A (FIG. 4A). Despite performing in silico analysis using two different platforms, TrueDesign Genome Editor™ and Cas-OFFinder, the off-target edit within the sequenced amplicon was not predicted. There were no unwanted edits within the sequenced amplicons when using gRNA for the remaining mutations.

### In Silico Analysis of Potential Off-Target Edits

In silico analysis revealed distinct off-target profiles for the three TSC2 mutations examined. The gRNA targeting P1 TSC2.1864C>T exhibited the most extensive off-target potential, with 18 predicted sites. The highest-scoring off-target site (CFD=0.459) was identified within the RORC intronic region. The majority of predicted off-target sites for this mutation were in intronic regions of various genes, with some additional sites in intergenic regions. These off-target sequences predominantly featured AGG PAM sequences, though other variants were also observed.

In contrast, the gRNA for P2 TSC2.1832 G>A demonstrated higher specificity, with only a single predicted off-target site located at chr15[55863032]. This off-target sequence (TGCAAATGGCAAGCAGCATC—SEQ ID NO: 33) contained mismatches at positions 4, 8, and 11, with a TGG PAM sequence. Most notably, the gRNA designed for P3 TSC2.5024 C>T showed the highest specificity, with no predicted off-target sites identified in the analysis.

### Delivery of CRISPR-Cas9 Using Electroporation

First, the SNP mutation P1 TSC2.1864C>T (p.Arg622Trp) was introduced into HEK293 cells using Lonza 4D, resulting in an editing score of 59% (FIG. 3). Using the same CRISPR components and hMSC-friendly device parameters, the 3 point mutations were targeted separately in hMSCs. One day after electroporation, all electroporated hMSCs showed a mean absorbance of 0.23 and mean cell viability of 39.7%. Over a 7-day observation, no significant cell recovery was detected (data not shown). To compare, several electroporation deliveries were performed using the Neon NxT system, targeting the same mutations using hMSC-specific conditions. On day 1 after the transfection, cells showed a mean absorbance of 0.13 and a mean cell viability without any improvement over 7 days after electroporation. Due to the low cell yield and viability, DNA extraction from all electroporated hMSCs was impossible, so CRISPR editing could not be confirmed.

Cell Viability and Proliferation after Lipofection-Based Transfection of RNPs for Base-Editing of TSC2 Gene in hMSCs

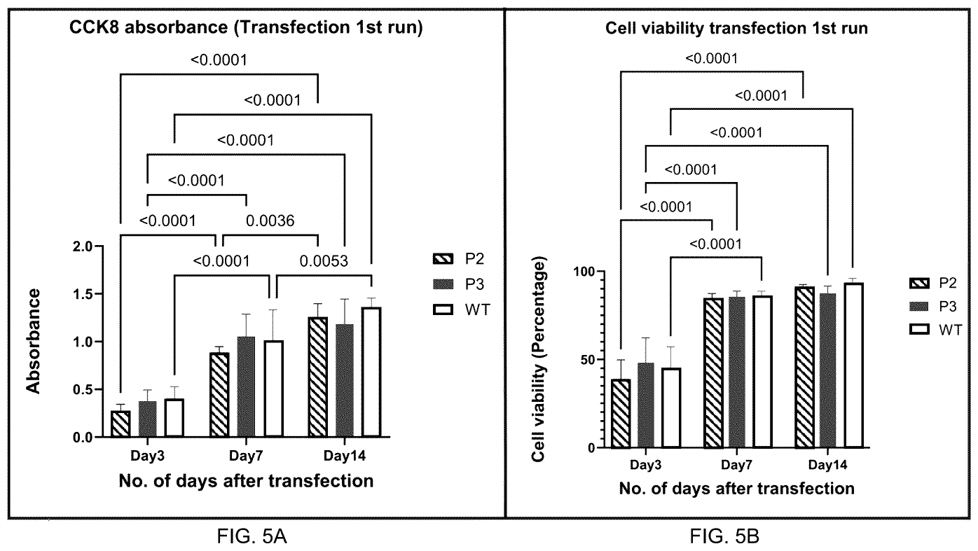

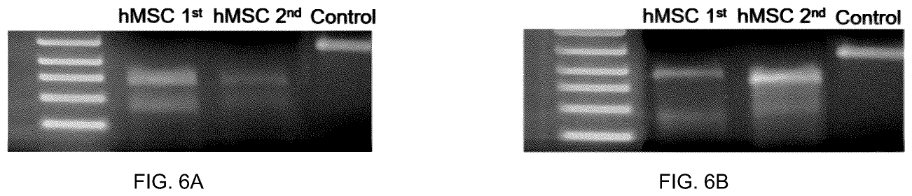

No morphological changes were observed in HEK293 cells or hMSCs in any of the successfully transfected cells, registering an average cell diameter of 50 μm. The CCK8 assay of the first transfection experiment with 500 ng gRNA showed a mean absorbance of 0.43 on day 3, 1.09 on day 7, and 1.27 on day 14 (FIG. 5A). The ANOVA showed significant differences in the mean absorbance in each cell group between day 3, day 7, and day 14. No significant differences were observed between the different cell groups (FIG. 5A). The cells had an average cell viability of 46.82% on day 3, 84.04% on day 7, and 92.33% on day 14. Similarly, differences in mean cell viability were observed in each cell group over the course of the 14 days, without any significant differences in mean cell viability between the cell groups (FIG. 5B). In the second transfection run with 750 ng of gRNA, cells showed an average absorbance in the CCK8 assay of 0.51 on day 3, 1.3 on day 7, and 1.27 on day 14 after transfection. The mean cell viability of 48.31% on the third day after transfection, 80.17% on day 7, and 89.94%. There were no significant differences observed in CCK8 absorbance or cell viability. The GeneArt Genomic Cleavage Detection Kit showed positive cleavage of the TSC2 gene in both base-editing experiments for P2 TSC2.1832 G>A (p.Arg611Glu) and P3 TSC2.5024 C>T (p.Pro1675Leu) compared to negative control hMSCs (FIGS. 6A-6B).

### Discussion

The first successful application of CRISPR-Cas9-based base editing to induce single nucleotide point mutations in TSC2 in mesenchymal stem cells (MSCs) was introduced herein. The editing efficiency of hMSCs matched and even exceeded that of the traditional positive control HEK293 cells. Similar efficiencies in hMSCs and HEK293 cells have been observed after GFP mRNA transfection. This precise modification of TSC2 in hMSCs could hold transformative potential for unfolding the hypothesized vital role of TSC2 in the initiation and progression of various cancerous and non-cancerous diseases. Furthermore, a workflow is presented herein that opens up potential avenues for developing cell-based therapies using genetically edited MSCs with minimal off-target effects, significantly enhancing the prospects of precision medicine in the treatment of genetic disorders, especially those related to stem cells. In particular, MSCs can be easily isolated and stored from Wharton's jelly and cord blood, which is a waste of delivery, but holds neonatal genomic material. There is a long history of hMSC engineering and transplantation; therefore, the current study has significant pre-clinical value and prospects for clinical translation.

Despite reaching a high editing efficiency of up to 85%, variation in editing efficiency was observed across the different targeted SNP mutations disparate from in silico predictions, as well as between the cell types. This common observation has been reported as a hurdle in most CRISPR-based base editing approaches. This can be attributed to several factors, including the delivery method of CRISPR components, specific target locus, chromatin accessibility of the gene, and repair machinery of the edited cells. Furthermore, variations in the efficiency of the base editors themselves, such as cytosine or adenine base editors (CBE or ABE), can lead to different outcomes depending on the nature of the base change being introduced. This variability underscores the need for careful optimization of CRISPR base editing protocols, including delivery systems, guide RNA design, and choice of base editor and mutation, to maximize efficiency and minimize off-target effects for specific application.

It is the inventors' belief that they are the first to report CRISPR-Cas9 base editing of TSC2 in hMSCs. Advantageously, unlike the methods of the prior art, wherein CRISPR knockout edits caused deletions of large portions of the DNA within the TSC2, the method described herein results in a nucleotide swap (i.e., single point mutation), with a negligible alteration of the TSC2 genomic sequence. This results in a better resemblance of the human pathology.

Given their high sensitivity to physical stress, gene-editing approaches for hMSCs require extensive design. This begins with choosing the gene of interest, the type of mutation targeted, delivery methods, and post-editing maintenance of mutagenesis. Despite previous reports of successful CRISPR-Cas9 delivery in mesenchymal stem cells using electroporation, our attempts have shown that cell viability is severely reduced after nucleofection despite using dedicated MSC protocols [Mun et al., 2016; Madeira et al., 2011]. Another disadvantage of electroporation is its limited clinical application for in vivo delivery of CRISPR-Cas9 components. Nonetheless, electroporation remains an effective gene delivery method, often with a trade-off between high cytotoxicity and reduced viability for specific cell types or if conditions are not carefully controlled.

On the other hand, Lipofectamine-based transfection has emerged as a widely adopted method for cellular cargo delivery, owing to its operational simplicity, cost-effectiveness, and compatibility with high-throughput screening systems. Both Lipofectamine 2000 and CRISPRMax are considered adequate for gene delivery, but differ significantly in their ability to deliver CRISPR components. CRISPRMax, designed explicitly for CRISPR/Cas9 delivery, has been optimized to minimize cytotoxicity while maintaining a high transfection efficiency in primary cells.

This example presents the successful CRISPR-Cas9 base editing of the TSC2 gene in hMSCs using Lipofectamine CRISPRMax. Although hMSCs showed significantly reduced cell viability within the first 3 days after transfection, complete cell recovery was observed within 14 days after the initial transfection. However, balancing the high CRISPR editing efficiency and low lipofectamine cytotoxicity remains important for achieving effective genetic modifications.

The efficacy of CRISPR-based base editing depends on the editing score, which is quantified through gene sequencing. In this example, Sanger sequencing was employed for the initial evaluation of editing scores, mainly because of its simplicity, cost-effectiveness, and ability to confirm the introduction of targeted base edits at specific loci. While this methodology presents inherent limitations in detecting mosaicism, off-target effects, and low-frequency editing events due to its restricted sensitivity and depth of analysis, it provided sufficient validation for the primary objective: demonstrating the feasibility of TSC2 gene base editing in human mesenchymal stem cells. Next-generation sequencing (NGS) overcomes these technical constraints by enabling comprehensive, high-throughput analysis across the genome, facilitating the detection of both on-target and off-target modifications. The superior resolution of NGS technology permits identification of rare editing events and mosaic patterns, which is important when precise characterization of editing outcomes are crucial for therapeutic development.

### CONCLUSION

CRISPR-based base editing shows high precision and efficiency for SNP modifications in hMSCs while maintaining cell viability. The successful editing of the TSC2 gene advances both gene editing technology and cancer research, enabling accurate modeling of TSC2-related pathologies. While Lipofectamine CRISPRMax proved effective for CRISPR-Cas9 delivery, an unpredicted off-target mutation was identified, highlighting the importance of experimental validation beyond computational predictions. Wharton's jelly and cord blood-derived hMSCs serve as excellent non-invasive sources for validating genome editing, with combining Sanger and next-generation sequencing providing comprehensive validation. The demonstrated efficiency in editing TSC2 mutations establishes a foundation for mutation studies in primary hMSCs, offering a more physiologically relevant system compared to derived cell lines.

Although the invention has been disclosed herein in details with reference to various embodiments and features, it will be appreciated by a person with ordinary skill in the art that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Barrangou R and Doudna J A. Applications of CRISPR technologies in research and beyond. Nat. Biotechnol. 2016; 34(9):933-941.
Caban C, Khan N, Hasbani D M, Crino P B. Genetics of tuberous sclerosis complex: implications for clinical practice. Appl Clin Genet. 2017; 10:1-8.
Cebrian-Serrano A and Davies B. CRISPR-Cas orthologues and variants: optimizing the repertoire, specificity and delivery of genome engineering tools. Mamm Genome. 2017; 28(7):247-261.
Chakraborty S, Mohiyuddin S M, Gopinath K S, Kumar A. Involvement of TSC genes and differential expression of other members of the mTOR signaling pathway in oral squamous cell carcinoma. BMC Cancer. 2008; 8:163.
Gornicka-Pawlak E, Janowski M, Habich A, Jablonska A, Sypecka J, Lukomska B. Intra-arterial Administration of Human Umbilical Cord Blood Derived Cells Inversed Learning Asymmetry Resulting From Focal Brain Injury in Rat. Front Neurol. 2019; 10:786.
Mehta M S, Vazquez A, Kulkarni D A, Kerrigan J E, Atwal G, Metsugi S, et al. Polymorphic variants in TSC1 and TSC2 and their association with breast cancer phenotypes. Breast Cancer Res Treat. 2011; 125(3):861-8.
Madeira C, Ribeiro S C, Pinheiro I S, Martins S A, Andrade P Z, da Silva C L, et al. Gene delivery to human bone marrow mesenchymal stem cells by microporation. J Biotechnol. 2011; 151(1):130-6.
Maeder M L and Gersbach C A. Genome-editing Technologies for Gene and Cell Therapy. Mol. Ther. 2016; 24(3):430-46.
Mun J Y, Shin K K, Kwon O, Lim Y T, Oh D B. Minicircle microporation-based non-viral gene delivery improved the targeting of mesenchymal stem cells to an injury site. Biomaterials. 2016; 101:310-20.
Slowinska M, Jozwiak S, Peron A, Borkowska J, Chmielewski D, Sadowski K, et al. Early diagnosis of tuberous sclerosis complex: a race against time. How to make the diagnosis before seizures? Orphanet J Rare Dis. 2018; 13(1):25.
Uysal S P, Sahin M. Tuberous sclerosis: a review of the past, present, and future. Turk J Med Sci. 2020; 50(SI-2):1665-76.
Wang S, Zhang C, Xu Z, Chen M H, Yu H, Wang L, et al. Differential impact of PI3K/AKT/mTOR signaling on tumor initiation and progression in animal models of prostate cancer. Prostate. 2023; 83(1):97-108.

What is claimed is:

1. A method for generating genomically engineered cells, the method comprising:
(a) providing a pool of sample cells;
(b) introducing into the pool of sample cells a Cas component, a guide RNA (gRNA) directed to a gRNA target, and a homology directed repair (HDR) template to produce a pool comprising modified cells;
(c) culturing the pool comprising modified cells to produce a cultured pool; and
(d) selecting genomically engineered cells from the cultured pool wherein at least 35% of cells in the cultured pool comprise a mutation in a Tuberous sclerosis complex 2 (TSC2) gene.

2. The method of claim 1, wherein the mutation in the TSC2 gene is in a Cyclin-B1 binding domain and GAP domain.

3. The method of claim 1, wherein the mutation in the TSC2 gene comprises at least one substitution of a first cytosine to a first thymine at nucleic acid position 5024 (5024C>T), a guanine to an adenine at position 1832 (1832G>A), a second cytosine to a second thymine at position 1864 (1864C>T), or any combination thereof.

4. The method of claim 1, wherein the Cas component comprises a Cas9 protein.

5. The method of claim 1, wherein the gRNA is designed by using a platform to target the mutation in the TSC2 gene, wherein the gRNA is selected based on a highest editing efficiency.

6. The method of claim 1, wherein the gRNA is designed by using a platform to target the mutation in the TSC2 gene, wherein the gRNA is selected based on a lowest off-target efficiency.

7. The method of claim 1, wherein the gRNA target comprises a sequence having at least 90% sequence identity to the sequence of SEQ ID NO: 1, SEQ ID NO: 2, or SEQ ID NO: 3.

8. The method of claim 1, wherein the HDR template comprises a sequence having at least 90% sequence identity to the sequence of SEQ ID NO: 4, SEQ ID NO: 5, or SEQ ID NO: 6.

9. The method of claim 1, wherein the sample cells comprise a mesenchymal stem cell.

10. The method of claim 1, further comprising transfecting the Cas component, the gRNA, and HDR template into the pool of sample cells with a lipid-based transfection agent.

11. The method of claim 10, wherein the lipid-based transfection agent comprises a lipofectamine agent.

12. The method of claim 11, wherein the lipofectamine agent is Lipofectamine 2000.

13. The method of claim 11, wherein the lipofectamine agent is Lipofectamine CRISPRMAX.

14. The method of claim 1, further comprising assaying cell viability of the genomically engineered cells, wherein at least 60% of cells in the pool of genomically engineered cells are viable after fourteen days.

15. The method of claim 1, wherein the genomically engineered cells comprise at least one nucleotide point mutation.

16. A method for correcting or reversing a pathogenic mutation in a TSC2 gene, the method comprising:
(a) providing a pathogenic cell comprising the pathogenic mutation at the TSC2 gene;
(b) introducing into the pathogenic cell a Cas component, a guide RNA (gRNA) directed to a gRNA target, and a corrective homology directed repair (HDR) template to produce a corrected cell,
wherein the corrective HDR template comprises a nonpathogenic sequence of the TSC2 gene.

17. The method of claim 16, wherein the pathogenic mutation is a single nucleotide point mutation.
